(19) United States
(12) Patent Application Publication
(10) Pub. No.: US 2025/0336539 A1
(43) Pub. Date: Oct. 30, 2025

(54) Generation of Health-Related Statistical Insights and Visualizations

(71) Applicant: Google LLC, Mountain View, CA (US)

(72) Inventors: Kent Edward Eisenhuth, Wynnewood, PA (US); Alex Shyng-Yueh Lin, San Jose, CA (US); Naghmeh Rezaei, Redwood City, CA (US); Katharine Ann Normandin, Brighton, MA (US); Matthew George Morrison, Phoenix, AZ (US); Joscelin Cooper, Oakland, CA (US); Dominique Sum-Yhee Meury, San Mateo, CA (US)

(21) Appl. No.: 18/649,282

(22) Filed: Apr. 29, 2024

(51) Int. Cl. G16H 50/30; G06N 20/00; G16H 10/60; G16H 50/20

(52) U.S. Cl. CPC G16H 50/30; G06N 20/00; G16H 10/60; G16H 50/20

(57) ABSTRACT

Methods, systems, devices, and non-transitory computer readable media for processing health data are provided. The disclosed technology can include receiving queries associated with health data comprising health metrics. Based on inputting the queries into machine-learned models, objectives associated with the queries and the health data can be determined. Statistical insights based on the objectives and health data can be determined. Furthermore, key indications based on the statistical insights and the objectives can be generated. The key indications can comprise visualizations associated with at least one of the health metrics.

### FIELD

The present disclosure relates generally to processing health data. More particularly, the present disclosure relates to the use of machine-learned models to parse natural language queries and generate statistical insights and visualizations based on the queries.

### BACKGROUND

Computing devices can be configured to use sensors to detect physical states of a user and process sensor values associated with the physical states. The sensor values associated with these physical states can be used to generate a variety of information including information associated with the health conditions of a user. Further, the information associated with the physical states can be accessed via a user interface that allows a user to perform operations including selecting certain information and changing the way that information is presented. However, certain users may find some types of user interfaces or user interface interactions to be difficult or too complex to effectively navigate. As such, there can be different ways to organize information that is related to the physical states of a user.

### SUMMARY

Aspects and advantages of embodiments of the present disclosure will be set forth in part in the following description, or can be learned from the description, or can be learned through practice of the embodiments.

One example aspect of the present disclosure is directed to a computer-implemented method of processing health data. The computer-implemented method can comprise receiving, by a computing system comprising one or more processors, one or more queries associated with health data comprising a plurality of health metrics. The computer-implemented method can comprise determining, by the computing system, based on inputting the one or more queries into one or more machine-learned models, one or more objectives associated with the one or more queries. The computer-implemented method can comprise determining, by the computing system, one or more statistical insights based on the one or more objectives and the health data. The computer-implemented method can comprise generating, by the computing system, one or more key indications based on the one or more statistical insights and the health data. The one or more indications can comprise one or more visualizations associated with at least one health metric of the plurality of health metrics.

Another example aspect of the present disclosure is directed to one or more tangible non-transitory computer-readable media storing computer-readable instructions that when executed by one or more processors cause the one or more processors to perform operations. The operations can comprise receiving one or more queries associated with health data comprising a plurality of health metrics. The operations can comprise determining, based on inputting the one or more queries into one or more machine-learned models, one or more objectives associated with the one or more queries. The operations can comprise determining one or more statistical insights based on the one or more objectives and the health data. The operations can comprise generating one or more key indications based on the one or more statistical insights and the health data. The one or more key indications can comprise one or more visualizations associated with at least one health metric of the plurality of health metrics.

Another example aspect of the present disclosure is directed to a computing system comprising: one or more processors; one or more non-transitory computer-readable media storing instructions that when executed by the one or more processors cause the one or more processors to perform operations. The operations can comprise receiving one or more queries associated with health data comprising a plurality of health metrics. The operations can comprise determining, based on inputting the one or more queries into one or more machine-learned models, one or more objectives associated with the one or more queries. The operations can comprise determining one or more statistical insights based on the one or more objectives and the health data. The operations can comprise generating one or more key indications based on the one or more statistical insights and the health data. The one or more key indications can comprise one or more visualizations associated with at least one health metric of the plurality of health metrics.

Other aspects of the present disclosure are directed to various systems, apparatuses, non-transitory computer-readable media, user interfaces, and electronic devices.

These and other features, aspects, and advantages of various embodiments of the present disclosure will become better understood with reference to the following description and appended claims. The accompanying drawings, which are incorporated in and constitute a part of this specification, illustrate example embodiments of the present disclosure and, together with the description, serve to explain the related principles.

### BRIEF DESCRIPTION OF THE DRAWINGS

Detailed discussion of embodiments directed to one of ordinary skill in the art is set forth in the specification, which makes reference to the appended figures, in which:

FIG. 1A depicts a block diagram of an example computing system that performs operations associated with the generation of statistical insights according to example embodiments of the present disclosure.

FIG. 1B depicts a block diagram of an example computing device that performs operations associated with the generation of statistical insights according to example embodiments of the present disclosure.

FIG. 1C depicts a block diagram of an example computing device that performs operations associated with the generation of statistical insights according to example embodiments of the present disclosure.

FIG. 2 depicts a block diagram of an example statistical insight generation computing device according to example embodiments of the present disclosure.

FIG. 3 depicts an example of a computing device according to example embodiments of the present disclosure.

FIG. 4 depicts an example of a user interface for statistical insight generation according to example embodiments of the present disclosure.

FIG. 5 depicts an example of a user interface for statistical insight generation according to example embodiments of the present disclosure.

FIG. 6 depicts an example of a user interface for statistical insight generation according to example embodiments of the present disclosure.

FIG. 7 depicts a flow chart diagram of an example method to perform operations associated with the generation of statistical insights according to example embodiments of the present disclosure.

FIG. 8 depicts a flow chart diagram of an example method to perform operations associated with the generation of statistical insights according to example embodiments of the present disclosure.

Reference numerals that are repeated across plural figures are intended to identify the same features in various implementations.

### DETAILED DESCRIPTION

In general, the present disclosure is directed to generating unique visualizations that provide rich information based on processing the health data of a user (e.g., the health data of a user of a wearable computing device). In particular, the disclosed technology can generate statistical insights that describe relationships (e.g., correlations between different health metrics) in health data based on objectives determined from a query (e.g., a user query with respect to the user's health data). Further, the disclosed technology can use machine-learned models (e.g., large language models (LLMs)) to parse the queries (e.g., natural language requests), thereby facilitating the generation of relevant statistical insights based on the user's queries and health data.

For example, a user can send a query associated with the user's health data to a computing system (e.g., a health data computing system) that is configured to receive and process such queries. The query can, for example, take the form of a request from the user for information regarding relationships between a user's heart rates and the user's sleep. The computing system that processes the query can implement a machine-learned model that is configured and/or trained to parse the query and determine objectives (e.g., the purpose of the query and/or the type of information the query is seeking) associated with the query. For example, the objectives can comprise a statistical analysis technique that can be performed on a portion of the health metrics (e.g., the health metrics that are relevant to the query). The objectives can be used to analyze the health data and generate statistical insights that can indicate relationships between various health metrics (e.g., the user's heart rate and sleep patterns). For example, the computing system can generate a statistical insight that indicates that an earlier bedtime may be correlated with lower average heart rates the next day. Further, the computing system can generate a recommendation for the user that is based on the statistical insight (e.g., a recommendation for a user to go to bed before a particular time in the evening).

The disclosed technology can then generate key indications that comprises descriptions associated with the statistical insight and/or visualizations that facilitate a user's understanding of the statistical insight. For example, the disclosed technology can be used to generate a description of the statistical insight that is accompanied by an infographic showing the relationship between lower average heart rates and earlier bedtime. As such, the disclosed technology allows for improved processing of health data in which a user can receive relevant statistical insights that are based on queries from the user and personalized based on the user's own health data. The disclosed technology therefore processes health data in a way that provides a user or healthcare professional with enhanced understanding of the health data as well as highlighting statistical relationships that might not otherwise be apparent.

Accordingly, the disclosed technology can improve the user's health improvement interactions by providing statistical insights into the user's health that are based on specific user queries. Further, the disclosed technology can assist a user in more effectively and/or safely performing the technical task of health data processing by means of a continued and/or guided human-machine interaction process in which queries are received and the disclosed technology generates statistical insights to assist the user's health data processing based on the received queries. The disclosed technology allows the generation of deep statistical insights that are tailored to a particular user's queries.

The disclosed technology can be implemented in a computing system (e.g., a health data computing system) that is configured to access data, perform operations on the data (e.g., parse queries, generate objectives, generate statistical insights, generate recommendations, and generate key indications that can comprise visualizations and are based on the statistical insights. Further, the computing system can leverage one or more machine-learned models that have been configured and/or trained to generate a variety of outputs objectives, statistical insights, recommendations, and key indications. The computing system can be included in a wearable computing device (e.g., a smartwatch or smart ring), mobile device (e.g., a smartphone or laptop computing device), and/or as part of a system that includes a server computing device that receives data associated with a queries about a user's health data from a user's client computing device (e.g., the user's smartwatch or smart phone), performs operations based on the data and sends output comprising indications (e.g., visualizations) based on statistical insights, and/or recommendations associated with a user's health data back to the client computing device. In some embodiments, the computing system can include specialized hardware and/or software that enables the performance of operations specific to the disclosed technology. For example, the computing system can include one or more application specific integrated circuits that are configured to perform operations associated with the generation of statistical insights and key indications that can assist a user in the task of processing health data.

The computing system can receive, access, and/or retrieve one or more queries. For example, the computing system can receive one or more queries via a user interface of a wearable device (e.g., a smartwatch) or a mobile device (e.g., a smartphone or laptop computing device). Further, the one or more queries can be based on text input (e.g., a text-based request for health information), image input (e.g., an image of a hand-written request for health information), and/or audio input (e.g., a spoken request for health information that is recognized and/or transcribed by a computing system).

The one or more queries can be associated with health data (e.g., a request for information associated with health data of a user) and/or a plurality of health metrics (e.g., heart rate metrics, sleep duration metrics, and/or caloric intake metrics) included in the health data. For example, the one or more queries can comprise a query associated with determining whether there is a relationship between multiple health metrics (e.g., “IS THERE A CONNECTION BETWEEN MY CALORIC INTAKE AND MY BLOOD PRESSURE?”), a query associated with relationships between a plurality of different health metrics over some time interval (e.g., “IS THERE A CORRELATION BETWEEN MY CALORIC INTAKE AND SLEEP QUALITY OVER THE LAST WEEK?”), a query associated with a comparison between a plurality of health metrics comprising different health metrics (e.g., “COMPARE MY LONG DISTANCE ROWING PERFORMANCE TO MY LONG DISTANCE RUNNING PERFORMANCE.”) or the same health metric at different time intervals (e.g., “COMPARE MY LONG DISTANCE ROWING PERFORMANCE IN MAY TO MY LONG DISTANCE ROWING PERFORMANCE IN JUNE.”), a query associated with trends relating to at least one health metric of the plurality of health metrics (e.g., “HOW HAS MY SLEEP QUALITY CHANGED OVER THE LAST MONTH?”), a query associated with the achievement of health goals (e.g., “HAS EATING LESS RESULTED IN WEIGHT LOSS?”), a query associated with determining an optimal or sub-optimal health outcome associated with a health metric (e.g., “IN THE PAST YEAR, IN WHAT MONTHS DID I HAVE THE HIGHEST AND LOWEST SLEEP QUALITY?”), and/or a query associated with the identification of maximal values in health metrics (e.g., “WHAT WAS MY HIGHEST BLOOD PRESSURE LAST MONTH?”), and/or a query associated with the identification of minimal values in health metrics (e.g., “WHAT WAS MY LOWEST BLOOD PRESSURE LAST MONTH?”).

The health data can comprise data associated with a user's health (e.g., a plurality of health metrics) and/or other data associated with the user (e.g., the user's name, age, and/or gender). The health data can comprise user reported goals (e.g., weight loss goals and/or fitness goals) and/or health logs and/or journal entries that are added to the health data by a user. The plurality of health metrics can comprise health benchmarks which can include personal baselines and ranges (e.g., mean and median fitness ranges), demographics (e.g., age and gender by peer cohort, which can be divided into percentile rankings), and/or health guidance ranges (e.g., recommended health ranges which can include recommended body mass ranges (BMI) for different groups). The plurality of health metrics can comprise heart rates over time, resting heart rate, maximum heart rate (e.g., the maximum heart rate during intense exercise), heart rate variability, oxygen saturation (SpO₂), skin temperature, breathing rate, blood pressure, caloric intake (e.g., daily caloric intake), sleep duration (e.g., sleep duration in hours), bedtime, mass (e.g., body mass in kilograms), and/or step count (e.g., daily step count). Further, the plurality of health metrics can comprise a plurality of heart rates at a plurality of time intervals, a plurality of body mass values at a plurality of time intervals, a plurality of sleeping hours associated with a plurality of time intervals, and/or a number of steps associated with a plurality of time intervals.

The plurality of health metrics can be based on health data from one or more sources (e.g., one or more sensors which can include different types of sensors). Further, the health data can be based on a user's performance of one or more physical activities. For example, the health metrics can comprise running times (e.g., 1500 meter running times), the mass of a maximum deadlift, rowing times (e.g., 2000-meter rowing times or rowing ergometer times), and/or cycling times (e.g., 10 kilometer cycling times). Further, the health data can comprise health metrics based on the number of movements of a physical activity that can be performed in some predetermined time interval (e.g., a maximum number of pull ups in one minute and/or a maximum number of jump squats that can be performed in one minute). The one or more physical activities may be included in the one or more queries and the health metrics associated with the one or more physical activities and may be used in the generation of the one or more objectives, one or more statistical insights, and/or one or more key indications.

The computing system can determine and/or generate one or more objectives. The one or more objectives can be associated with the one or more queries and/or one or more types of information (e.g., at least one health metric of the plurality of health metrics and/or one or more statistical analysis techniques) associated with the one or more queries and/or the health data. Further, the one or more objectives can comprise one or more statistical analysis techniques to perform on one or more health metrics selected from the plurality of health metrics. For example, if the one or more queries indicate “DOES JOGGING AFFECT MY SLEEP QUALITY” the computing system can determine that the objective of the query is to perform one or more statistical analysis techniques to determine one or more relationships between the health metrics associated with running (e.g., “jogging”) and the health metrics associated with sleep (e.g., “sleep quality”).

Determination of the one or more objectives can comprise parsing the one or more queries which can include determining one or more portions of the one or more queries that correspond to one or more semantic units (e.g., individual words and/or phrases that carry meaning) and/or determining a structure (e.g., a syntactic structure) of the one or more queries. The one or more semantic units that are determined and/or the syntactic structure of the one or more queries can be used to determine one or more portions of the one or more queries that are associated with health data (e.g., health metrics that are associated with the one or more queries).

Further, determining the one or more objectives can comprise identifying one or more key terms (e.g., key words or key phrases) in the one or more queries. For example, based on the query “DOES MY STRESS GO DOWN WHEN I WALK?” the computing system can determine that the one or more key terms comprise the words “STRESS,” “GO DOWN,” and “WALK.” In some embodiments, determination of the one or more key terms can be based on the use of one or more natural language processing techniques. For example, the computing system can be configured to use one or more natural language processing techniques to determine the context of the one or more queries, one or more synonyms of the one or more key terms, and/or one or more portions of the one or more queries that are inferred and not directly stated. The computing system can then search the health data to determine one or more health metrics of the plurality of metrics in the health data that are associated with the one or more key terms. In the example of a query with the words “STRESS” and “WALK,” the computing system can determine that the one or more health metrics associated with the one or more queries comprise the user's blood pressure metrics (e.g., a health metric that can be associated with the “stress” key term) and the steps taken metric (e.g., a health metric that can be associated with the “walk” key term).

Determining the one or more objectives can comprise classifying one or more portions of the one or more queries. Classification of the one or more portions of the one or more queries can be based on use of one or more machine-learned models that are configured and/or trained to classify one or more queries and generate output comprising one or more health metrics that are relevant to the one or more queries and one or more statistical analysis techniques to perform on the one or more relevant health metrics selected from the plurality of health metrics. For example, the one or more queries can be classified as: queries for comparisons of different health metrics, queries for comparisons of the same health metric at different time intervals, queries to identify anomalous health metrics, queries to identify trends associated with health metrics, queries for correlations associated with health metrics, queries for health metric variability over time, queries for the deviation of one or more user health metrics from mean health metric values (e.g., mean health metric values of the user and/or other users) over time, a query for a ranked list associated with a health metric (e.g., a ranked list of months with the highest number of steps or longest sleep duration), and/or queries to identify changes in health metrics that are attributable to other health metrics.

In some embodiments, the computing system can determine the one or more objectives based on inputting the one or more queries into one or more machine-learned models. For example, the computing system can input the one or more queries and/or the health data into one or more machine-learned models that are configured and/or trained to determine the one or more objectives based on input comprising the one or more queries and/or the health data. The one or more machine-learned models can comprise one or more large language models (LLMs) that are configured to determine the one or more objectives based on identifying health-related information in the one or more queries. Further, the one or more machine-learned models can be configured and/or trained to process natural language inputs (e.g., text-based input, image-based input (e.g., an image of a handwritten query), and/or audio-based input). The one or more machine-learned models can generate one or more objectives comprising one or more statistical techniques to perform on one or more health metrics selected from the plurality of health metrics based on the one or more queries (e.g., relevant health metrics that are directly and/or indirectly indicated in the one or more queries).

The one or more machine-learned models can be trained to determine one or more objectives based on training data that can comprise generalized language data (e.g., non-health related books and general interest articles), health specific language data (e.g., medical texts and medica articles), and/or personalized data (e.g., text that was generated based on a user associated with a particular set of health data). Further, the one or more machine-learned models can be trained and/or retrained based on additions to health data (e.g., current health data), new types of health data (e.g., new types of health data based on new sensor types of sensor outputs), and/or one or more changes in the existing health data (e.g., the modification and/or deletion of health data).

The computing system can determine and/or generate one or more statistical insights based on the one or more objectives which can be associated with the one or more queries. The computing system can use the one or more objectives to determine one or more statistical analysis techniques to use on the health data in order to determine the one or more statistical insights. For example, if the one or more objectives are associated with determining relationships between the plurality of health metrics comprising sleep metrics and blood pressure metrics, the computing system can determine that one or more correlation techniques can be used to determine correlations between the sleep metrics and the blood pressure metrics.

The one or more statistical analysis techniques can comprise determining one or more mean values of at least one health metric of the plurality of health metrics (e.g., daily mean morning resting heart rate values over a one-month time interval), determining a standard deviation associated with at least one health metric of the plurality of health metrics, correlating two or more health metrics of the plurality of health metrics, and/or performing regression analysis (e.g., linear regression analysis) on at least one health metric of the plurality of health metrics. Based on use of the one or more statistical analysis techniques on the health data, the computing system can determine the one or more statistical insights (e.g., one or more statistical insights that are relevant to the one or more objectives).

In some embodiments, the one or more queries, the one or more objectives, and/or the health data can be used as part of an input to one or more machine-learned models that are configured and/or trained to receive the input, perform one or more operations on the input, and generate an output comprising the one or more statistical insights. Further, the one or more machine-learned models can be configured to determine the one or more statistical insights based on using the one or more objectives to select one or more statistical analysis technique (e.g., correlation of two health metrics) and/or perform one or more statistical analysis techniques on the health data (e.g., determine a correlation between two health metrics). Further, the one or more machine-learned models can be configured and/or trained to generate the one or more statistical insights in the form of natural language. The one or more machine-learned models can generate one or more statistical insights that describe correlations in colloquial terms (e.g., a statistical insight can indicate “AN EARLIER BEDTIME CAN RESULT IN A LOWER RESTING HEART RATE”). The one or more machine-learned models can be trained using training data that can comprise one or more training objectives, one or more training health metrics, and one or more training statistical insights. Further, the one or more machine-learned models can be trained and/or retrained based on a user's use of the one or more machine-learned models.

Determining the one or more statistical insights can comprise determining one or more relationships between at least two health metrics of the plurality of health metrics. For example, the computing system can determine one or more changes in one or more health metrics that are associated with one or more changes in one or more other health metrics. Determining the one or more statistical insights can comprise the use of simple linear regression to determine the strength of relationships between two health metrics or multiple linear regression to determine the strength of relationships between a health metric and two or more other health metrics. For example, the computing system can use linear regression to determine the strength of a relationship between steps taken by a user and the user's bedtime. By way of further example, the one or more relationships can comprise one or more correlations between two different health metrics (e.g., correlations between blood pressure and steps taken).

The computing system can determine that the one or more statistical insights comprise relationships between health metrics in which the strength of the relationship between the health metrics exceeds a relationship threshold. Health metrics in which the relationship between the health metrics is below the relationship threshold may not be included in the one or more statistical insights. For example, if the strength of a correlation between health metrics comprising steps taken and caloric intake is below the relationship threshold, then the correlation may not be included in the one or more statistical insights and another statistical insight in which a correlation between two health metrics is stronger (e.g., exceeds the relationship threshold) may be used.

The one or more statistical insights can comprise a trend associated with at least one health metric of the plurality of health metrics. For example, the trend can indicate a direction in which a health metric is trending (e.g., an increase in steps taken, a decrease in body mass, or no change in average resting heart rate). Further, the one or more statistical insights can indicate the magnitude of a trend. For example, the one or more statistical insights can indicate whether there is a strong upward trend or a slight downward trend.

The computing system can generate one or more key indications. The one or more key indications can be based on the one or more statistical insights. For example, if the one or more statistical insights are associated with health metrics comprising sleep duration and caloric intake, the one or more key indications can be associated with sleep duration and caloric intake. Further, the one or more key indications can comprise one or more descriptions (e.g., text-based descriptions indicating sleep durations and caloric intakes at a plurality of time intervals) and/or images (e.g., a line graph showing sleep durations and caloric intakes at a plurality of time intervals) based on the one or more statistical insights. In some embodiments, the generation of the one or more key indications may comprise determining the one or more key indications based on a significance (e.g., statistical significance) of the one or more health metrics associated with the one or more statistical insights. The health metrics that are included in the one or more key indications can be positively correlated with the significance of the health metrics such that more statistically significant health metrics are more likely to be included in the one or more key indications than less statistically significant health metrics.

Generating the one or more key indications can be based on use of one or more machine-learned models. The one or more queries, the one or more objectives, the one or more statistical insights, and/or the health data can be used as part of an input to one or more machine-learned models that are configured and/or trained to receive the input, perform one or more operations on the input, and generate an output comprising the one or more key indications, one or more key visualizations, and/or headline. Further, the one or more machine-learned models can be configured and/or trained to generate the one or more key indications in the form of natural language. For example, the one or more machine-learned models can generate one or more key indications in informal terms (e.g., a statistical insight can indicate “TAKE MORE THAN FIVE THOUSAND STEPS PER DAY”). The one or more machine-learned models can be trained using training data that can comprise one or more training objectives, one or more training health metrics, and one or more training statistical insights.

Further, the one or more key indications can comprise one or more visualizations associated with at least one health metric of the plurality of health metrics. The one or more visualizations can include text (e.g., words and/or numbers) and/or pictures that can be generated on a display device. Further, the visualizations can comprise one or more charts (e.g., area charts, bar charts, line charts, punchcard charts, and/or scatter plots), one or more graphs, one or more heatmaps, one or more histograms, and/or one or more infographics. For example, if the one or more statistical insights are associated with health metrics comprising sleep duration and caloric intake, the one or more key indications can comprise text and/or images that correspond to the health metrics (e.g., a line graph showing a relationship between sleep duration and caloric intake).

In some embodiments, the one or more infographics can comprise one or more images, one or more icons, and/or one or more portions of text. Further, one or more of the health metrics indicated in the one or more visualizations can be emphasized by using one or more indications (e.g., larger fonts and/or brighter colors). For example, in an infographic that shows a ranking of months based on steps taken, the month with the highest number of steps can be more brightly colored than other months indicated in the infographic.

The one or more key indications can comprise a text-based description of the one or more statistical insights. For example, the one or more key indications can comprise a natural language description that indicates one or more health metrics of the plurality of health metrics that are significant and can comprise comparisons, trends, and/or relationships between the plurality of health metrics. Further, the one or more key indications can comprise a description of the one or more relationships between at least two health metrics of the plurality of health metrics. For example, the one or more key indications can comprise a description of a correlation between two health metrics (e.g., a correlation between health metrics comprising sleep durations and running times).

In some embodiments, the one or more key indications can comprise one or more audio indications. For example, the one or more key indications can comprise one or more audio tones (e.g., musical tones) that can be associated with statistical insights. Further, the one or more key indications can comprise an audio-based description of the one or more statistical insights. For example, the one or more audio indications can comprise a synthetic voice that is used to indicate the statistical insights, the recommendations, and/or to describe the one or more visualizations (e.g., a synthetic voice describing the axes of a line graph, the types of health metrics indicated in the line chart, and/or the way in which the health metrics change over time).

The one or more key indications can comprise a scatter plot that indicates one or more relationships between at least two health metrics of the plurality of health metrics. For example, the computing system can generate a scatter plot graph that indicates the relationship between the plurality of health metrics comprising the heart rate of a user and the hours of exercise that a user performs. The scatter plot can indicate whether there is a positive or negative relationship between the at least two of the plurality of health metrics.

The one or more key indications can comprise one or more audio indications based on a type of the trend. For example, the one or more audio indications can comprise a musical tone that changes based on the type of trend. The one or more audio indications can comprise a first audio indication based on the type of the trend being an upward trend, a second audio indication based on the type of the trend being a horizontal trend, or a third audio indication based on the type of the trend being a downward trend. For example, if a health metric comprising steps taken per week and the health metric is trending upwards, the one or more audio indications can comprise cheerful musical tones. However, if the health metric is trending downwards then the one or more audio indications can comprise somber musical tones. In some embodiments, the one or more audio indications can comprise a synthetic voice that announces the direction of a trend associated with at least one health metric of the plurality of health metrics.

Generating the one or more key indications can comprise determining a headline that summarizes the one or more statistical insights. For example, the computing system can determine one or more key terms that can be used to summarize the one or more statistical insights. The computing system can then generate a headline that comprises the one or more key terms. Further, determining the headline can comprise inputting the one or more key indications into one or more machine-learned models that are configured to generate output comprising the headline.

Further, generating the one or more key indications can comprise generating the headline in a prominent location relative to the one or more visualizations. For example, the headline can be generated in a location that is prominent (e.g., above the one or more visualizations and/or below the one or more visualizations).

Generating the one or more key indications based on one or more statistical insights can comprise determining a recommendation that corresponds to at least one statistical insight of the one or more statistical insights. Further, the one or more key indications can be based on the recommendation. In some embodiments, the one or more queries, the one or more objectives, and/or the health data can be used as part of an input to one or more machine-learned models that are configured and/or trained to receive the input, perform one or more operations on the input, and generate an output comprising the recommendation. Further, the one or more machine-learned models can be configured and/or trained to generate the recommendation in the form of natural language. The one or more machine-learned models can generate a recommendation in informal terms (e.g., a statistical insight can indicate “TAKE MORE THAN FIVE THOUSAND STEPS PER DAY”). The one or more machine-learned models can be trained using training data that can comprise one or more training objectives, one or more training health metrics, and one or more training statistical insights.

The computing system can generate one or more secondary indications that are based on the one or more key indications. The one or more secondary indications can have a higher level of granularity (e.g., a greater number of indications relating to the same type of health data than the one or more key indications for the same time interval) than the one or more key indications and/or can have a greater depth of information than the one or more key indications. For example, if the one or more key indications comprise weekly health metrics (e.g., steps taken in a week and/or average resting heart rate for a week) over a twelve week period (e.g., twelve sets of steps taken in a week and twelve sets of the average resting heart rate for a week), the one or more secondary indications can comprise the same key indications (e.g., steps taken in a week and/or average resting heart rate for a week) on a more granular daily basis. Further, the one or more secondary indications can comprise the same key indications over a longer six-month period.

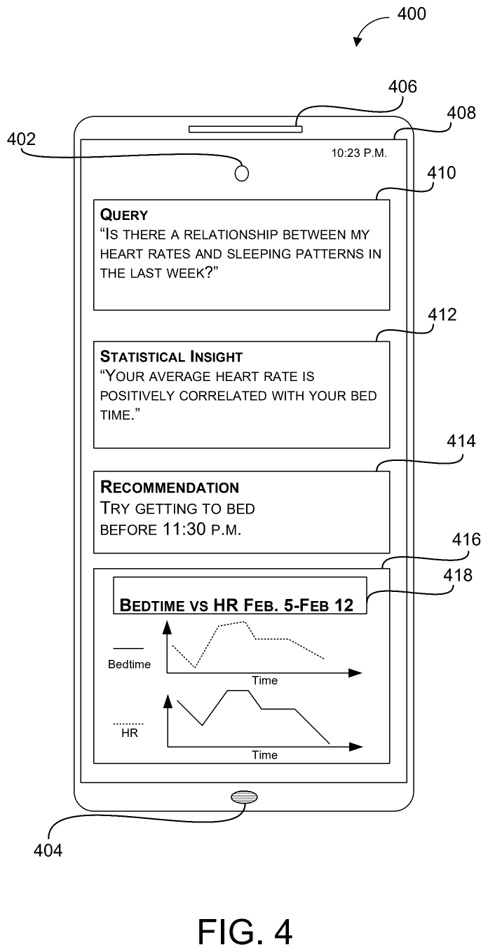

In some embodiments, the one or more secondary indications can be generated in response to receiving an input (e.g., an input from a user comprising a request for additional information associated with the one or more key indications). For example, a user can interact with (e.g., touch) an interface element of a user interface generated on a computing device (e.g., the interface generated on the computing device 400 that is described with respect to FIG. 4), which can cause the computing device to generate the one or more secondary indications.

The systems, methods, devices, computer-readable media (e.g., tangible non-transitory computer-readable media) in the disclosed technology can provide a variety of technical effects and benefits including an improvement in the generation of relevant statistical insights from health data. In particular, the disclosed technology may assist a user (e.g., a user of a health processing device) in performing technical tasks by means of a continued and/or guided human-machine interaction process in which health data can be continuously updated and queried to determine statistical insights.

Further, the disclosed technology can provide the technical effect of improving the effectiveness with which health related tasks are performed. For example, the computing system can be continuously updated based on user queries and/or changes in the user's health data, thereby allowing for more relevant statistical insights to be provided to the user. For example, machine-learned models that are used as part of the process of generating statistical insights can be continuously trained and/or updated in response to queries from a user. This has the effect of providing more relevant and accurate statistical insights related to a particular user's health data. Further, based on the generation of statistical insights the disclosed technology can identify potential health issues and alert users about those issues.

The disclosed technology may improve the operation of a health processing device by more effectively performing a variety of tasks with the specific benefits of improving health outcomes and/or alerting a user of potential health issues. Further, the specific benefits provided to users can be used to improve the effectiveness of a wide range of devices and services health monitoring devices and health monitoring services. Accordingly, the improvements offered by the disclosed technology can result in tangible benefits to a variety of devices and/or systems comprising computing systems, electronic systems, and/or mechanical systems associated with health data processing.

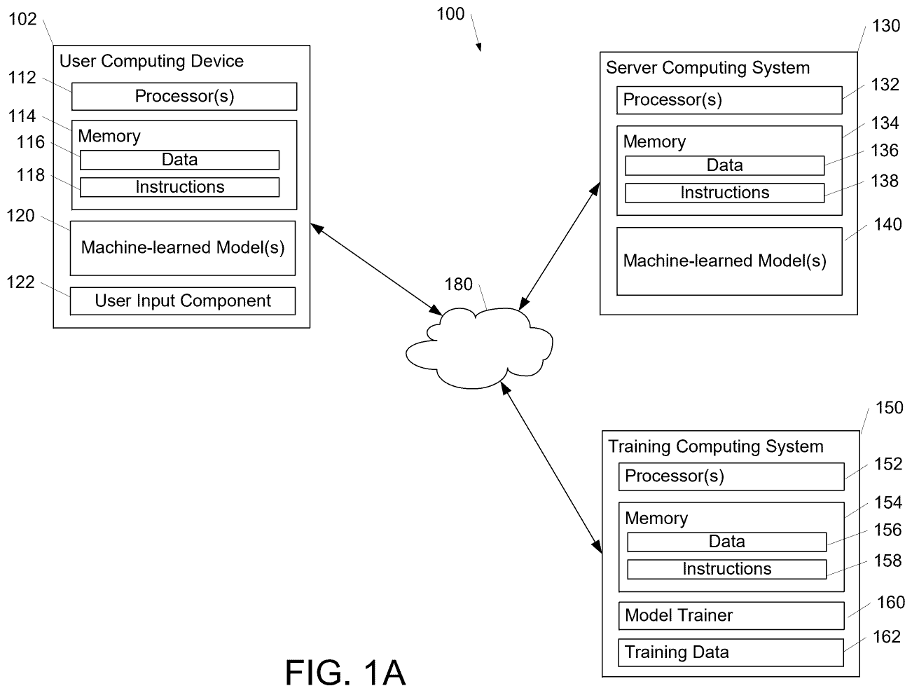

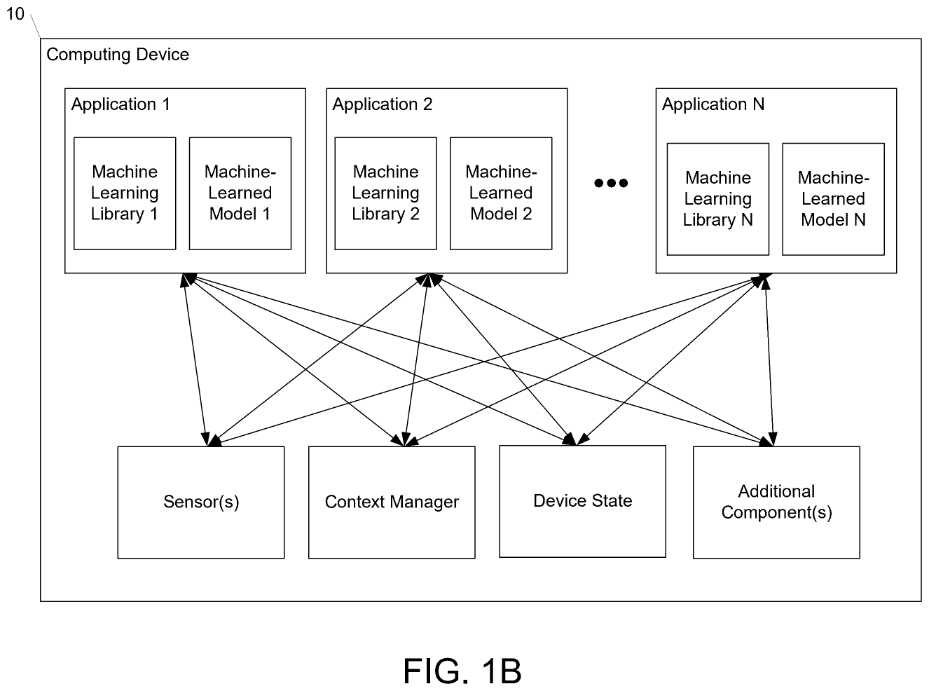

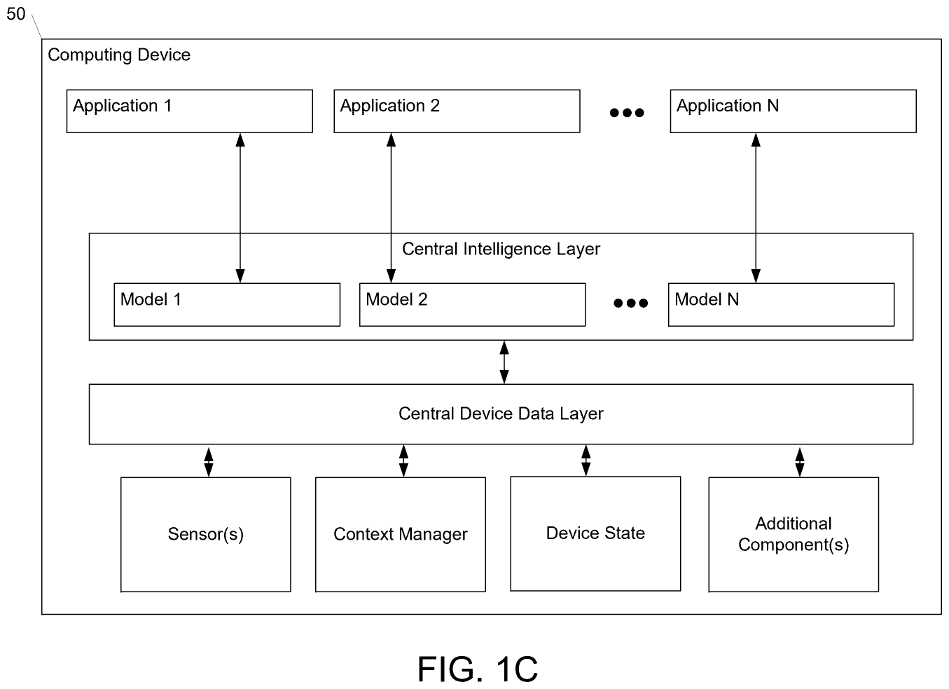

With reference now to the figures, example embodiments of the present disclosure will be discussed in further detail. FIG. 1A depicts a block diagram of an example of a computing system 100 that performs operations associated with the generation of statistical insights according to example embodiments of the present disclosure. The system 100 includes a computing device 102, a server computing system 130, and a training computing system 150 that are communicatively coupled over a network 180.

The computing device 102 can comprise any type of computing device, such as, for example, a wearable computing device (e.g., a smartwatch), a personal computing device (e.g., laptop or desktop), a mobile computing device (e.g., smartphone or tablet), a gaming console or controller, an embedded computing device, or any other type of computing device.

The computing device 102 includes one or more processors 112 and a memory 114. The one or more processors 112 can be any suitable processing device (e.g., a processor core, a microprocessor, an ASIC, an FPGA, a controller, a microcontroller, etc.) and can be one processor or a plurality of processors that are operatively connected. The memory 114 can include one or more non-transitory computer-readable storage media, such as RAM, ROM, EEPROM, EPROM, flash memory devices, magnetic disks, etc., and combinations thereof. The memory 114 can store data 116 and instructions 118 which are executed by the processor 112 to cause the computing device 102 to perform operations.

In some implementations, the computing device 102 can store or include one or more machine-learned models 120. For example, the one or more machine-learned models 120 can be or can otherwise include various machine-learned models such as neural networks (e.g., deep neural networks) or other types of machine-learned models, comprising non-linear models and/or linear models. Neural networks can include feed-forward neural networks, recurrent neural networks (e.g., long short-term memory recurrent neural networks), convolutional neural networks or other forms of neural networks. Some example machine-learned models can leverage an attention mechanism such as self-attention. For example, some example machine-learned models can include multi-headed self-attention models (e.g., transformer models). Examples of one or more machine-learned models 120 are discussed with reference to FIGS. 1-8.

In some implementations, the one or more machine-learned models 120 can be received from the server computing system 130 over network 180, stored in the user computing device memory 114, and then used or otherwise implemented by the one or more processors 112. In some implementations, the computing device 102 can implement multiple parallel instances of a single machine-learned model 120 (e.g., to perform parallel statistical insight generation operations across multiple instances of the one or more machine-learned models 120).

More particularly, the one or more machine-learned models 120 can comprise one or more machine-learned models (e.g., one or more large language models) that are configured to parse queries (e.g., text-based queries and/or audio-based queries) in order to determine health metrics that are associated with the query and/or health data.

Additionally or alternatively, one or more machine-learned models 140 can be included in or otherwise stored and implemented by the server computing system 130 that communicates with the computing device 102 according to a client-server relationship. For example, the one or more machine-learned models 140 can be implemented by the server computing system 130 as a portion of a web service (e.g., a statistical insight generation service). Thus, one or more machine-learned models 120 can be stored and implemented at the computing device 102 and/or one or more machine-learned models 140 can be stored and implemented at the server computing system 130.

The computing device 102 can also include one or more user input components 122 that receives user input. For example, the user input component 122 can be a touch-sensitive component (e.g., a touch-sensitive display screen or a touch pad) that is sensitive to the touch of a user input object (e.g., a finger or a stylus). The touch-sensitive component can serve to implement a virtual keyboard. Other example user input components include a microphone, a traditional keyboard, or other means by which a user can provide user input.

The server computing system 130 includes one or more processors 132 and a memory 134. The one or more processors 132 can be any suitable processing device (e.g., a processor core, a microprocessor, an ASIC, an FPGA, a controller, a microcontroller, etc.) and can be one processor or a plurality of processors that are operatively connected. The memory 134 can include one or more non-transitory computer-readable storage media, such as RAM, ROM, EEPROM, EPROM, flash memory devices, magnetic disks, etc., and combinations thereof. The memory 134 can store data 136 and instructions 138 which are executed by the processor 132 to cause the server computing system 130 to perform operations.

In some implementations, the server computing system 130 includes or is otherwise implemented by one or more server computing devices. In instances in which the server computing system 130 includes plural server computing devices, such server computing devices can operate according to sequential computing architectures, parallel computing architectures, or some combination thereof.

As described above, the server computing system 130 can store or otherwise include one or more machine-learned models 140. For example, the one or more machine-learned models 140 can be or can otherwise include various machine-learned models. Example machine-learned models include neural networks or other multi-layer non-linear models. Example neural networks include feed forward neural networks, deep neural networks, recurrent neural networks, and convolutional neural networks. Some example machine-learned models can leverage an attention mechanism such as self-attention. For example, some example machine-learned models can include multi-headed self-attention models (e.g., transformer models). Examples of one or more machine-learned models 140 are discussed with reference to FIGS. 1-8.

The computing device 102 and/or the server computing system 130 can train the one or more machine-learned models 120 and/or the one or more machine-learned models 140 via interaction with the training computing system 150 that is communicatively coupled over the network 180. The training computing system 150 can be separate from the server computing system 130 or can be a portion of the server computing system 130.

The training computing system 150 includes one or more processors 152 and a memory 154. The one or more processors 152 can be any suitable processing device (e.g., a processor core, a microprocessor, an ASIC, an FPGA, a controller, a microcontroller, etc.) and can be one processor or a plurality of processors that are operatively connected. The memory 154 can include one or more non-transitory computer-readable storage media, such as RAM, ROM, EEPROM, EPROM, flash memory devices, magnetic disks, etc., and combinations thereof. The memory 154 can store data 156 and instructions 158 which are executed by the processor 152 to cause the training computing system 150 to perform operations. In some implementations, the training computing system 150 includes or is otherwise implemented by one or more server computing devices.

The training computing system 150 can include a model trainer 160 that trains the one or more machine-learned models 120 and/or the one or more machine-learned models 140 stored at the computing device 102 and/or the server computing system 130 using various training or learning techniques, such as, for example, backwards propagation of errors. For example, a loss function can be backpropagated through the model(s) to update one or more parameters of the model(s) (e.g., based on a gradient of the loss function). Various loss functions can be used such as mean squared error, likelihood loss, cross entropy loss, hinge loss, and/or various other loss functions. Gradient descent techniques can be used to iteratively update the parameters over a number of training iterations.

In some implementations, performing backwards propagation of errors can include performing truncated backpropagation through time. The model trainer 160 can perform a number of generalization techniques (e.g., weight decays, dropouts, etc.) to improve the generalization capability of the models being trained.

In particular, the model trainer 160 can train the one or more machine-learned models 120 and/or the one or more machine-learned models 140 based on a set of training data 162. The training data 162 can include, for example, health data and/or other data that is associated with the health, physical state of a user, and/or activities (e.g., physical activities and/or food consumption) performed by a user. For example, the training data can comprise heart rate data (e.g., the heart rate of a user at one or more times), mass data (e.g., the mass in kilograms of a user at one or more times), age data (e.g., the age of a user), blood pressure data (e.g., the blood pressure of a user at one or more times), sleep data (e.g., the sleep duration of a user at one or more times and/or the bedtimes of a user), and/or step data (e.g., the number of steps a user takes on one or more days or during one or more activities).

In some implementations, if the user has provided consent, the training examples can be provided by the computing device 102. Thus, in such implementations, the one or more machine-learned models 120 provided to the computing device 102 can be trained by the training computing system 150 on user-specific data received from the computing device 102. In some instances, this process can be referred to as personalizing the model.

The model trainer 160 includes computer logic utilized to provide desired functionality. The model trainer 160 can be implemented in hardware, firmware, and/or software controlling a general-purpose processor. For example, in some implementations, the model trainer 160 includes program files stored on a storage device, loaded into a memory, and executed by one or more processors. In other implementations, the model trainer 160 includes one or more sets of computer-executable instructions that are stored in a tangible computer-readable storage medium such as RAM, hard disk, or optical or magnetic media.

The network 180 can be any type of communications network, such as a local area network (e.g., intranet), wide area network (e.g., Internet), or some combination thereof and can include any number of wired or wireless links. In general, communication over the network 180 can be carried via any type of wired and/or wireless connection, using a wide variety of communication protocols (e.g., TCP/IP, HTTP, SMTP, FTP), encodings or formats (e.g., HTML, XML), and/or protection schemes (e.g., VPN, secure HTTP, SSL).

The machine-learned models described in this specification may be used in a variety of tasks, applications, and/or use cases. In some implementations, the input to the machine-learned model(s) of the present disclosure can be text or natural language data. The machine-learned model(s) can process the text or natural language data to generate an output (e.g., based on inputting queries from a user the machine-learned model(s) can process and generate one or more objectives associated with the queries and health data of the user). As an example, the machine-learned model(s) can process the natural language data to generate a language encoding output. As another example, the machine-learned model(s) can process the text or natural language data to generate a latent text embedding output. As another example, the machine-learned model(s) can process the text or natural language data to generate a translation output. As another example, the machine-learned model(s) can process the text or natural language data to generate a classification output. As another example, the machine-learned model(s) can process the text or natural language data to generate a textual segmentation output. As another example, the machine-learned model(s) can process the text or natural language data to generate a semantic intent output. As another example, the machine-learned model(s) can process the text or natural language data to generate an upscaled text or natural language output (e.g., text or natural language data that is higher quality than the input text or natural language, etc.). As another example, the machine-learned model(s) can process the text or natural language data to generate a prediction output.

In some implementations, the input to the machine-learned model(s) of the present disclosure can be speech data. The machine-learned model(s) can process the speech data to generate an output. As an example, the machine-learned model(s) can process the speech data to generate a speech recognition output. As another example, the machine-learned model(s) can process the speech data to generate a speech translation output. As another example, the machine-learned model(s) can process the speech data to generate a latent embedding output. As another example, the machine-learned model(s) can process the speech data to generate an encoded speech output (e.g., an encoded and/or compressed representation of the speech data, etc.). As another example, the machine-learned model(s) can process the speech data to generate an upscaled speech output (e.g., speech data that is higher quality than the input speech data, etc.). As another example, the machine-learned model(s) can process the speech data to generate a textual representation output (e.g., a textual representation of the input speech data, etc.). As another example, the machine-learned model(s) can process the speech data to generate a prediction output.

In some implementations, the input to the machine-learned model(s) of the present disclosure can be latent encoding data (e.g., a latent space representation of an input, etc.). The machine-learned model(s) can process the latent encoding data to generate an output. As an example, the machine-learned model(s) can process the latent encoding data to generate a recognition output. As another example, the machine-learned model(s) can process the latent encoding data to generate a reconstruction output. As another example, the machine-learned model(s) can process the latent encoding data to generate a search output. As another example, the machine-learned model(s) can process the latent encoding data to generate a reclustering output. As another example, the machine-learned model(s) can process the latent encoding data to generate a prediction output.

In some implementations, the input to the machine-learned model(s) of the present disclosure can be statistical data. Statistical data can be, represent, or otherwise include data computed and/or calculated from some other data source. The machine-learned model(s) can process the statistical data to generate an output. As an example, the machine-learned model(s) can process the statistical data to generate a recognition output. As another example, the machine-learned model(s) can process the statistical data to generate a prediction output. As another example, the machine-learned model(s) can process the statistical data to generate a classification output. As another example, the machine-learned model(s) can process the statistical data to generate a segmentation output. As another example, the machine-learned model(s) can process the statistical data to generate a visualization output. As another example, the machine-learned model(s) can process the statistical data to generate a diagnostic output.

In some implementations, the input to the machine-learned model(s) of the present disclosure can be sensor data. The machine-learned model(s) can process the sensor data to generate an output. As an example, the machine-learned model(s) can process the sensor data to generate a recognition output. As another example, the machine-learned model(s) can process the sensor data to generate a prediction output. As another example, the machine-learned model(s) can process the sensor data to generate a classification output. As another example, the machine-learned model(s) can process the sensor data to generate a segmentation output. As another example, the machine-learned model(s) can process the sensor data to generate a visualization output. As another example, the machine-learned model(s) can process the sensor data to generate a diagnostic output. As another example, the machine-learned model(s) can process the sensor data to generate a detection output.

In some cases, the machine-learned model(s) can be configured to perform a task that includes encoding input data for reliable and/or efficient transmission or storage (and/or corresponding decoding). For example, the task may be an audio compression task. The input may include audio data and the output may comprise compressed audio data. In another example, the input includes visual data (e.g., one or more images or videos), the output comprises compressed visual data, and the task is a visual data compression task. In another example, the task may comprise generating an embedding for input data (e.g., input audio or visual data).

In some cases, the input includes audio data representing a spoken utterance and the task is a speech recognition task. The output may comprise a text output which is mapped to the spoken utterance. In some cases, the task comprises encrypting or decrypting input data. In some cases, the task comprises a microprocessor performance task, such as branch prediction or memory address translation.

FIG. 1A illustrates one example computing system that can be used to implement the present disclosure. Other computing systems can be used as well. For example, in some implementations, the computing device 102 can include the model trainer 160 and the training data 162. In such implementations, the one or more machine-learned models 120 can be both trained and used locally at the computing device 102. In some of such implementations, the computing device 102 can implement the model trainer 160 to personalize the one or more machine-learned models 120 based on user-specific data.

FIG. 1B depicts a block diagram of an example of a computing device 10 that performs according to example embodiments of the present disclosure. The computing device 10 can be a user computing device or a server computing device.

The computing device 10 includes a number of applications (e.g., applications 1 through N). Each application contains its own machine-learned library and machine-learned model(s). For example, cach application can include a machine-learned model. Example applications include a text messaging application, an email application, a dictation application, a virtual keyboard application, a browser application, etc.

As illustrated in FIG. 1B, cach application can communicate with a number of other components of the computing device, such as, for example, one or more sensors, a context manager, a device state component, and/or additional components. In some implementations, cach application can communicate with each device component using an API (e.g., a public API). In some implementations, the API used by each application is specific to that application.

FIG. 1C depicts a block diagram of an example of a computing device 50 that performs according to example embodiments of the present disclosure. The computing device 50 can be a user computing device or a server computing device.

The computing device 50 includes a number of applications (e.g., applications 1 through N). Each application is in communication with a central intelligence layer. Example applications include a health application (e.g., an application that is used to process health data that can be based on sensor outputs from a wearable computing device of a user), text messaging application, an email application, a dictation application, a virtual keyboard application, a browser application, etc. In some implementations, each application can communicate with the central intelligence layer (and model(s) stored therein) using an API (e.g., a common API across all applications).

The central intelligence layer includes a number of machine-learned models. For example, as illustrated in FIG. 1C, a respective machine-learned model can be provided for each application and managed by the central intelligence layer. In other implementations, two or more applications can share a single machine-learned model. For example, in some implementations, the central intelligence layer can provide a single model for the applications. In some implementations, the central intelligence layer is included within or otherwise implemented by an operating system of the computing device 50.

The central intelligence layer can communicate with a central device data layer. The central device data layer can be a centralized repository of data for the computing device 50. As illustrated in FIG. 1C, the central device data layer can communicate with a number of other components of the computing device, such as, for example, one or more sensors, a context manager, a device state component, and/or additional components. In some implementations, the central device data layer can communicate with each device component using an API (e.g., a private API).

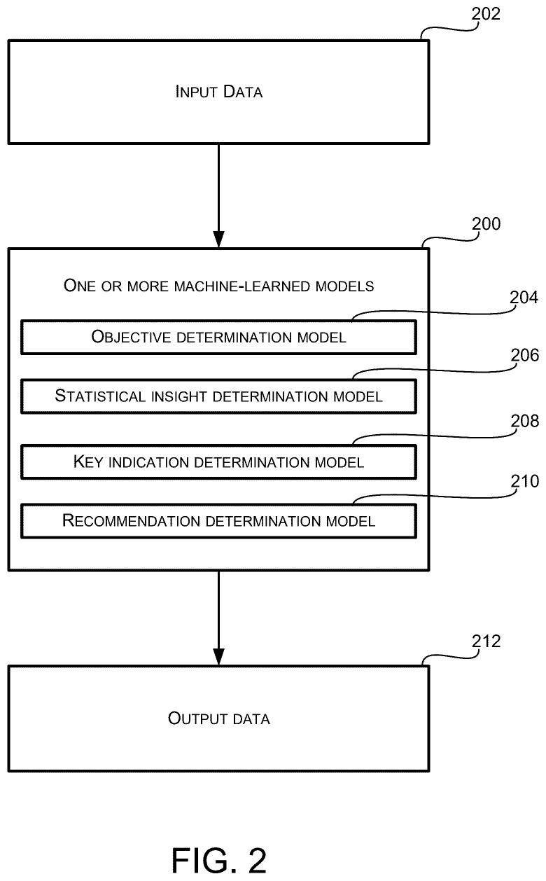

FIG. 2 depicts a block diagram of an example of one or more machine-learned models 200 according to example embodiments of the present disclosure. In some implementations, the one or more machine-learned models 200 is trained to receive a set of input data 202 descriptive of a query for information associated with health data and, as a result of receipt of the input data 202 (e.g., input data comprising health data, one or more queries, one or more objectives, one or more statistical insights, one or more key indications, one or more visualizations, and/or one or more recommendations), provide output data 212 that can comprise one or more objectives, one or more statistical insights, one or more key indications, one or more headlines, one or more visualizations, and/or one or more recommendations. In some implementations, the one or more machine-learned models 200 can include an objective generation model 204 that is operable to generate one or more objectives based on receiving input comprising one or more queries. In some implementations, the one or more machine-learned models 200 can include a statistical insight generation model 206 that is operable to generate one or more statistical insights based on receiving input comprising one or more objectives and/or health data. In some implementations, the one or more machine-learned models 200 can include a key indication generation model 208 that is operable to one or more key indications based on receiving input one or more statistical insights. In some implementations, the one or more machine-learned models 200 can include a recommendation determination model 210 that is operable to one or more recommendations based on receiving input comprising one or more statistical insights and/or health data.

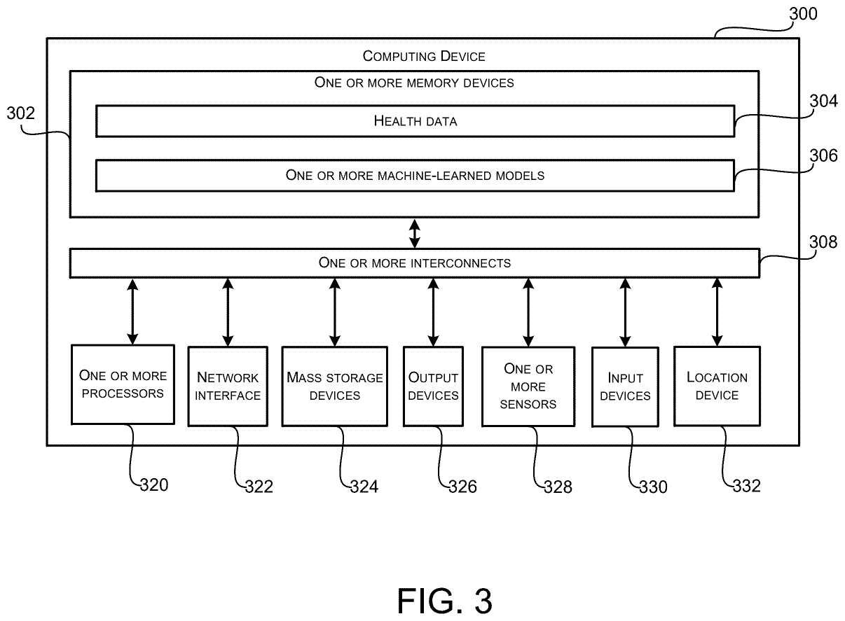

FIG. 3 depicts an example of a computing device according to example embodiments of the present disclosure. A computing device 300 can include one or more features and/or capabilities of the computing device 102, the server computing system 130, and/or the training computing system 150. Furthermore, the computing device 300 can perform one or more actions and/or operations performed by the computing device 102, the server computing system 130, and/or the training computing system 150, which are described with respect to FIG. 1A.

As shown in FIG. 3, the computing device 300 can include one or more memory devices 302, health data 304, one or more machine-learned models 306, one or more interconnects 308, one or more processors 320, a network interface 322, one or more mass storage devices 324, one or more output devices 326, one or more sensors 328, one or more input devices 330, and/or the location device 332. The computing device 300 can be configured as a wearable computing device (e.g., a smartwatch, fitness tracker, fitness band, or smart ring) and/or a mobile computing device (e.g., a smartphone, tablet computing device, and/or laptop computing device). Further, the computing device 300 can generate health data based on one or more states of a user (e.g., one or more physical states of a user that are detected by the one or more sensors 328 of the computing device 300), data that is received from another computing device (e.g., health data that is generated by a remote computing device), and/or data that is entered by a user (e.g., a user can enter information indicating the food consumed by the user).

The one or more memory devices 302 can store information and/or data (e.g., the health data 304 and/or the one or more machine-learned models 306). Further, the one or more memory devices 302 can include one or more computer-readable mediums (e.g., tangible non-transitory computer-readable media), including RAM, ROM, EEPROM, EPROM, flash memory devices, magnetic disks, and combinations thereof. The information and/or data stored by the one or more memory devices 302 can be executed by the one or more processors 320 to cause the computing device 300 to perform operations including operations associated with using one or more queries to determine one or more objectives, determine one or more statistical insights associated with health data, and generate one or more key indications.

The health data 304 can include one or more portions of data (e.g., the data 116, the data 136, and/or the data 156, which are depicted in FIG. 1A) and/or instructions (e.g., the instructions 118, the instructions 138, and/or the instructions 158 which are depicted in FIG. 1A) that are stored in the memory 114, the memory 134, and/or the memory 154, respectively. Furthermore, the health data 304 can include information associated with the health and/or physical state of a user (e.g., a user of the computing device 300). In some embodiments, the health data 304 can be received from one or more computing systems (e.g., the server computing system 130 that is depicted in FIG. 1) which can include one or more computing systems that are remote (e.g., in another city) from the computing device 300.

The health data 304 can include one or more portions of data (e.g., the data 116, the data 136, and/or the data 156, which are depicted in FIG. 1A) and/or instructions (e.g., the instructions 118, the instructions 138, and/or the instructions 158 which are depicted in FIG. 1A) that are stored in the memory 114, the memory 134, and/or the memory 154, respectively. Furthermore, the health data 304 can include information that was generated by the one or more sensors 328 of the computing device 300. For example, the health data 304 can comprise a plurality of heart rates detected by a heart rate monitor (e.g., an electrocardiogram) of the one or more sensors 328. In some embodiments, the health data 304 can be received from one or more computing systems (e.g., the server computing system 130 that is depicted in FIG. 1) which can include one or more computing systems that are remote from the computing device 300.

The one or more machine-learned models 306 (e.g., the one or more machine-learned models 120 and/or the one or more machine-learned models 140) can include one or more portions of the data 116, the data 136, and/or the data 156 which are depicted in FIG. 1A and/or instructions (e.g., the instructions 118, the instructions 138, and/or the instructions 158 which are depicted in FIG. 1A) that are stored in the memory 114, the memory 134, and/or the memory 154, respectively. Furthermore, the one or more machine-learned models 306 can include information associated with receiving data comprising one or more queries and/or health data comprising a plurality of health metrics; determining one or more objectives; determining one or more statistical insights; and generating output comprising one or more key indications associated with the one or more statistical insights. In some embodiments, the one or more machine-learned models 306 can be received from one or more computing systems (e.g., the server computing system 130 that is depicted in FIG. 1) which can include one or more computing systems that are remote from the computing device 300.

The one or more interconnects 308 can include one or more interconnects or buses that can be used to send and/or receive one or more signals (e.g., electronic signals) and/or data (e.g., the health data 304, and/or the one or more machine-learned models 306) between devices of the computing device 300, including the one or more memory devices 302, the one or more processors 320, the network interface 322, the one or more mass storage devices 324, the one or more output devices 326, the one or more sensors 328, and/or the one or more input devices 330. The one or more interconnects 308 can be arranged or configured in different ways, including as parallel or serial connections. Further the one or more interconnects 308 can include one or more internal buses to connect the internal components of the computing device 300; and one or more external buses used to connect the internal components of the computing device 300 to one or more external devices. By way of example, the one or more interconnects 308 can include different interfaces including Industry Standard Architecture (ISA), Extended ISA, Peripheral Components Interconnect (PCI), PCI Express, Serial AT Attachment (SATA), HyperTransport (HT), USB (Universal Serial Bus), Thunderbolt, IEEE 1394 interface (FireWire), and/or other interfaces that can be used to connect components.

The one or more processors 320 can include one or more computer processors that are configured to execute the one or more instructions stored in the one or more memory devices 302. For example, the one or more processors 320 can, for example, include one or more general purpose central processing units (CPUs), application specific integrated circuits (ASICs), and/or one or more graphics processing units (GPUs). Further, the one or more processors 320 can perform one or more actions and/or operations including one or more actions and/or operations associated with the health data 304 and/or the one or more machine-learned models 306. The one or more processors 320 can include single or multiple core devices including a microprocessor, microcontroller, integrated circuit, and/or a logic device.

The network interface 322 can support network communications. For example, the network interface 322 can support communication via networks including a local area network and/or a wide area network (e.g., the Internet). Further, the network interface 322 can be used to receive data (e.g., health data) from other computing devices. The one or more mass storage devices 324 (e.g., a hard disk drive and/or a solid-state drive) can be used to store data including the health data 304 and/or the one or more machine-learned models 306.

The one or more output devices 326 can include one or more display devices (e.g., LCD display, OLED display, Mini-LED display, microLED display, plasma display, and/or CRT display), one or more light sources (e.g., LEDs), one or more audio output devices (e.g., one or more loudspeakers), and/or one or more haptic output devices (e.g., one or more devices that are configured to generate vibratory output). For example, the one or more output devices 326 can comprise a touch sensitive display that is used to output an interface (e.g., a user interface) that can be configured to receive queries (e.g., text-based queries via the touch sensitive display) and generate indications comprising one or more key indications based on one or more statistical insights.

The one or more sensors 328 can comprise one or more heart rate sensors, one or more accelerometers, one or more gyroscopes, one or more altimeters, one or more temperature sensors, one or more bioimpedance sensors, one or more barometric pressure sensors, and/or one or more oximetry sensors. The one or more sensors 328 can be configured to generate sensor output comprising health data. For example, the one or more sensors 328 can generate health data comprising heart rate data based on sensor output from the one or more heart rate sensors. By way of further example, the one or more sensors 328 can generate health data comprising oxygen saturation (SpO₂) data based on sensor output from the one or more oximetry sensors. The one or more sensors 328 can be configured to generate health data based on the use of data from a single sensor (e.g., the accelerometer), multiple sensors of the one or more sensors 328 (e.g., the accelerometer and the gyroscope), and/or data from other devices (e.g., location data from the location device 332). For example, the one or more sensors 328 can generate health data comprising step metrics and/or distance walked metrics based on the use of data (e.g., location data) from the location device 332, the one or more accelerometers, and/or the one or more gyroscopes.

The one or more input devices 330 can include one or more keyboards, one or more touch sensitive devices (e.g., a touch screen display), one or more buttons (e.g., a power button and/or volume buttons), one or more microphones, and/or one or more imaging devices (e.g., one or more cameras).

The one or more memory devices 302 and the one or more mass storage devices 324 are illustrated separately, however, the one or more memory devices 302 and the one or more mass storage devices 324 can be regions within the same memory module. The computing device 300 can include one or more additional processors, memory devices, network interfaces, which may be provided separately or on the same chip or board. The one or more memory devices 302 and the one or more mass storage devices 324 can include one or more computer-readable media, including, but not limited to, non-transitory computer-readable media, RAM, ROM, hard drives, flash drives, and/or other memory devices.

The one or more memory devices 302 can store sets of instructions for applications including an operating system that can be associated with various software applications or data. For example, the one or more memory devices 302 can store sets of instructions for applications that can generate output including one or more statistical insights. The one or more memory devices 302 can be used to operate various applications including a mobile operating system developed specifically for mobile devices. As such, the one or more memory devices 302 can store instructions that allow the software applications to access data including data associated with the generation of one or more statistical insights associated with health data. In other embodiments, the one or more memory devices 302 can be used to operate or execute a general-purpose operating system that operates on both mobile and stationary devices, including for example, smartphones, laptop computing devices, tablet computing devices, and/or desktop computers.

The software applications that can be operated or executed by the computing device 300 can include applications associated with the system 100 shown in FIG. 1A. Further, the software applications that can be operated and/or executed by the computing device 300 can include native applications and/or web-based applications.

The location device 332 can include one or more devices or circuitry for determining the position of the computing device 300. For example, the location device 332 can determine an actual and/or relative position of the computing device 300 by using a satellite navigation positioning system (e.g., a GPS system, a Galileo positioning system, the GLObal Navigation satellite system (GLONASS), and/or the BeiDou Satellite Navigation and Positioning system), an inertial navigation system, a dead reckoning system, based on IP address, by using triangulation and/or proximity to cellular towers and/or Wi-Fi hotspots. The location device 332 can be used in the generation of health metrics including the steps taken by a user and/or a distance travelled by a user.

FIG. 4 depicts an example of a statistical insight generation system according to example embodiments of the present disclosure. A computing device 400 can include one or more features and/or capabilities of the computing device 102, the server computing system 130, the training computing system 150, and/or the computing device 300. Furthermore, the computing device 400 can perform one or more actions and/or operations that can be performed by the computing device 102, the server computing system 130, the training computing system 150, and/or the computing device 300.

As shown in FIG. 4, the computing device 400 includes an imaging component 402, an audio input component 404, an audio output component 406, a display component 408, a query 410, a statistical insight 412, a recommendation 414, a visualization 416, and a headline 418.

The computing device 400 can be configured to perform one or more operations comprising sending, receiving, processing, and/or generating data comprising health data and/or other data received by the computing device 400 (e.g., data associated with one or more queries). In some embodiments, the computing device 400 can comprise a mobile computing device (e.g., a smartphone and/or a wearable computing device) that can be configured to process data locally and/or receive data from a remote source (e.g., a remote computing device that stores and/or processed data comprising health data). The data (e.g., health data) received by the computing device 400 can be used to generate output comprising one or more statistical insights (e.g., the statistical insight 412) based on one or more queries (e.g., the query 410). Further, the computing device 400 can be configured to generate output comprising a recommendation (e.g., the recommendation 414) that is based on the one or more insights. The computing device 400 can also generate a visualization (e.g., visualization 416) that can comprise one or more images that support the information indicated in the statistical insight 412 and/or recommendation 414.

Further, the computing device 400 can implement an interface (e.g., a user interface) that is configured to receive one or more inputs (e.g., touch inputs and/or verbal inputs) from a user and perform operations which can comprise generating the statistical insight 412, the recommendation 414, the visualization 416, and/or the headline 418. In some embodiments, the computing device 400 can generate one or more audio indications (e.g., generating audio tones that indicate the achievement of health goals and/or a synthetic voice that speaks audio indications associated with the statistical insight 412) via the audio output component 406 (e.g., a loudspeaker) of the computing device 400.

In this example, the computing device 400 has received the query 410, which is displayed on the display component 408. The query 410 indicates “IS THERE A RELATIONSHIP BETWEEN MY HEART RATES AND SLEEPING PATTERNS IN THE LAST WEEK?”

In some embodiments, the computing device 400 can determine the identity of the user that sent the query 410 based on use of the imaging component 402 (e.g., a camera) that can be configured to capture an image of a user of the computing device 400. The computing device 400 can perform image recognition operations on the image to determine the identity of the user and, after further authenticating the user (e.g., biometric authentication and/or a passcode) and receiving the user's express authorization to access the user's health data, can securely access health data (e.g., encrypted health data) that is associated with that user.

Further, the computing device 400 can use the query 410 and/or health data associated with the query 410 as an input to one or more machine-learned models that can be implemented on the computing device 400 and/or that are implemented on a remote computing device that is able to send data to and/or receive data from the computing device 400. The one or more machine-learned models can be configured to parse the query 410 and generate output comprising one or more objectives that may be used to determine the statistical insight 412, the recommendation 414, the visualization 416, and/or the headline 418. Based on the query 410, the computing device 400 can determine that the query is requesting a correlation (e.g., “RELATIONSHIP”) that is related to the heart rate and sleeping patterns of the user within a specified time frame (e.g., “THE LAST WEEK”). The one or more machine-learned models implemented by the computing device 400 can generate one or more objectives that indicate which of one or more statistical analysis techniques (e.g., multiple linear regression) to perform on one or more health metrics selected from the plurality of health metrics.

Further, the computing device 400 can use the one or more objectives to generate one or more statistical insights. The one or more statistical insights can be based on performing the one or more statistical analysis techniques on the one or more health metrics determined from the query 410 and indicated in the one or more objectives. Based on the one or more objectives, the health data on which to perform the one or more statistical analysis techniques can comprise heart rates of the user (e.g., heart rates captured each minute), sleep durations (e.g., the number of hours a user sleeps per night), and/or bedtimes (e.g., the time the user went to sleep) over the time interval specified in the query 410 (e.g., February 5-12). For example, the computing device 400 can determine average heart rates of the user and one or more relationships between the heart rates of the user and the sleep durations and/or bedtimes of the user within the specified time interval (e.g., February 5-12).

The statistical insight 412 can be displayed on the display component 408 and can indicate the result of the statistical analysis of the query 410. In this example, the statistical insight 412 indicates “YOUR AVERAGE HEART RATE IS POSITIVELY CORRELATED WITH YOUR BEDTIME.” The statistical insight 412 can be based on the one or more relationships that were determined to be of high significance (e.g., the most significant correlation between heart rate and sleep patterns) and/or the one or more relationships comprising terms (e.g., words) that are most closely related to the query 410 (e.g., the term “SLEEP PATTERNS” may be determined by the one or more machine-learned models to be more closely related to bedtimes than sleep durations).

The recommendation 414 can be displayed on the display component 408 and can be based on statistical insight 412. In this example, the recommendation 414 indicates “TRY GETTING TO BED BEFORE 11:30 P.M.” The recommendation 414 can be based on the statistical insight 412, the one or more query objections, and/or the health data. The recommendation 414 can be based on one or more rules (e.g., one or more heuristics) that can be applied to the statistical insight 412, the one or more objectives, and/or the health data. For example, a heuristic can be used to identify a bedtime range that occurs when heart rates in the specified time interval are between a maximum heart rate and minimum average heart rate.

By way of further example, the recommendation 414 can be based on the use of one or more machine-learned models. For example, the statistical insight 412, the one or more objectives, and/or the health data can be inputted into one or more machine-learned modes, which can be configured to generate the recommendation 414 based on processing the statistical insight 412, the one or more objectives, and/or the health data. The one or more machine-learned models can be configured and/or trained based on use of training data comprising a plurality of training statistical insights and a plurality of ground-truth training recommendations. Over a plurality of training iterations, the one or more machine-learned models can be trained to generate more accurate recommendations. For example, training data comprising heart rate and sleep related health metrics can be used to determine accurate recommendations that comprise recommendations that are accurate (e.g., recommendations that are close to ground-truth results).

In some embodiments, the computing device 400 can generate the statistical insight 412 and/or the recommendation 414 via the audio output component 406. For example, the computing device 400 can generate a synthetic voice that reads the statistical insight 412 and/or the recommendation 414 via the audio output component 406. Further, the audio output component 406 can be used to indicate (e.g., via audio tones) a type of statistical insight or recommendation. For example, if a statistical insight indicates that a user is making progress towards a health goal (e.g., sleeping through the night without interruption), the computing device 400 can generate celebratory music. However, if the statistical insight indicates that a user is regressing with respect to achieving a health goal (e.g., sleeping less than eight hours per night), the computing device 400 can generate more somber toned music.

The computing device 400 can generate the visualization 416. The visualization 416 can be displayed on the display component 408 and can comprise one or more key indications based on the statistical insight 412. In this example, the visualization 416 comprises a line chart that shows the bedtime of the user over a plurality of days and another line chart that shows average heart rates of the user over a plurality of days. The charts indicated in the visualization 416 can comprise a horizontal axis that indicates time and vertical axes that indicate a bedtime or heart rate (HR). The visualization 416 can graphically represent some of the health data that the statistical insight 412 is based on. In some embodiments, the visualization 416 can be expanded to show additional information that is associated with the statistical insight 412. For example, the visualization 416 can be expanded to show the heart rates and/or bedtimes over a longer time interval that ranges beyond February 5-12. Further, the headline 418 can be generated to facilitate understanding of the visualization 416. The headline 418 can be generated based on the use of one or more machine-learned models. The one or more machine-learned models can be configured to use the statistical insight 412 as an input that is used to generate the headline 418 which comprises the one or more health metrics (e.g., heart rates, bedtimes, and time) that are significant and indicated in the statistical insight 412.

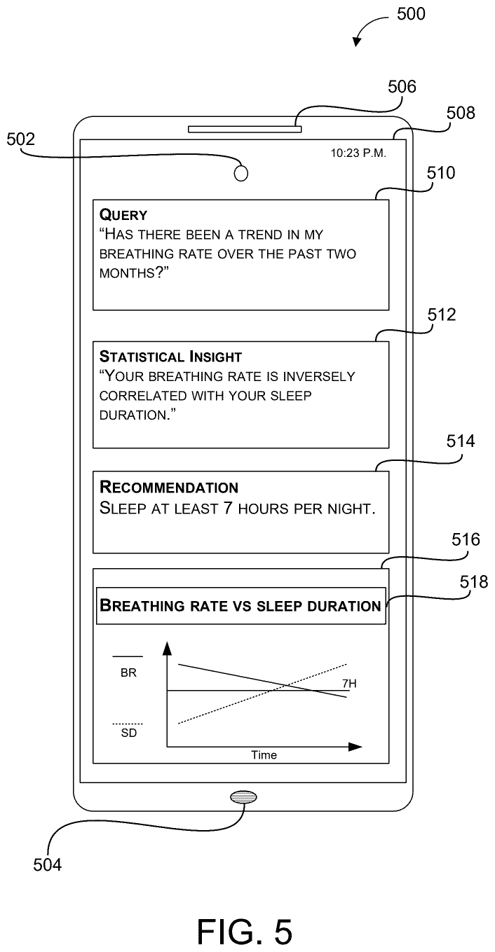

FIG. 5 depicts an example of a statistical insight generation system according to example embodiments of the present disclosure. A computing device 500 can include one or more features and/or capabilities of the computing device 102, the server computing system 130, the training computing system 150, the computing device 300, and/or the computing device 400. Furthermore, the computing device 500 can perform one or more actions and/or operations that can be performed by the computing device 102, the server computing system 130, the training computing system 150, and/or the computing device 300.

As shown in FIG. 5, the computing device 500 includes an imaging component 502, an audio input component 504, an audio output component 506, a display component 508, a query 510, a statistical insight 512, a recommendation 514, a visualization 516, and a headline 518. The computing device 500 can be configured to perform one or more operations comprising generating one or more key indications based on one or more statistical insights (e.g., statistical insights associated with steps taken by a user).

In this example, the computing device 500 has received the query 510, which is displayed on the display component 508. The query 510 indicates “HAS THERE BEEN A TREND IN MY BREATHING RATE OVER THE PAST TWO MONTHS?” The query can be from a user associated with the computing device 500. Based on the query 510, the computing device 500 can determine that the query 510 is seeking to determine whether there are trends in health metrics associated with the user's breathing rate within a two-month time interval. The computing device 500 can input the query 510 into one or more machine-learned models implemented by the computing device 500. The one or more machine-learned models can be configured to parse the query 510 and generate output comprising one or more objectives, the statistical insight 512, the recommendation 514, the visualization 516, and/or the headline 518. The one or more machine-learned models implemented by the computing device 400 can generate one or more objectives that indicate which of one or more statistical analysis techniques (e.g., trend analysis techniques) to perform on one or more health metrics selected from the plurality of health metrics.

Further, the computing device 500 can use the one or more objectives to generate one or more statistical insights. The one or more statistical insights can be based on performing the one or more statistical analysis techniques on the one or more health metrics determined from the query 510 and indicated in the one or more objectives. Based on the one or more objectives, the health data on which to perform the one or more statistical analysis techniques can comprise breathing rates of the user over the time interval specified in the query 510 (e.g., the past two months). Further, the computing device 500 can process the plurality of health metrics to determine which of the other health metrics has a strong relationship (e.g., the health metric changes significantly when the breathing rate changes) with the breathing rate. For example, the computing device 500 can determine that there is a relationship between the breathing rate and the sleep duration (e.g., the breathing rate is inversely correlated with sleep duration).

The statistical insight 512 can be displayed on the display component 508 and can indicate the result of the statistical analysis of the query 510. In this example, the statistical insight 512 indicates “YOUR BREATHING RATE IS INVERSELY CORRELATED WITH YOUR SLEEP DURATION.” The statistical insight 512 can be based on the health metric (e.g., sleep duration) that was determined to have the strongest relationship with the breathing rate of the user in the past two months.

The recommendation 514 can be displayed on the display component 508 and can be based on statistical insight 512. In this example, the recommendation 514 indicates “GET MORE SLEEP.” The recommendation 514 can be based on the statistical insight 512, the one or more objectives, and/or the health data. The recommendation 514 can be based on one or more rules (e.g., one or more heuristics) that can be applied to the statistical insight 512, the one or more objectives, and/or the health data. For example, a heuristic can be used to determine sleep durations that are associated with various breathing rates of the user. The computing device 500 can recommend that the user get more sleep if the high breathing rates occur after a user has slept less than some predetermined amount.

The computing device 500 can generate the visualization 516. The visualization 516 can be displayed on the display component 508 and can comprise one or more key indications based on the statistical insight 512. In this example, the visualization 516 comprises a line chart that shows the breathing rates and sleep duration of the user over a two-month period. Further, the visualization 516 indicates the breathing rate at the seven-hour sleep duration threshold. The visualization 516 can graphically represent some of the health data that the statistical insight 512 is based on. Further, the headline 518 can be generated to facilitate understanding of the visualization 516.

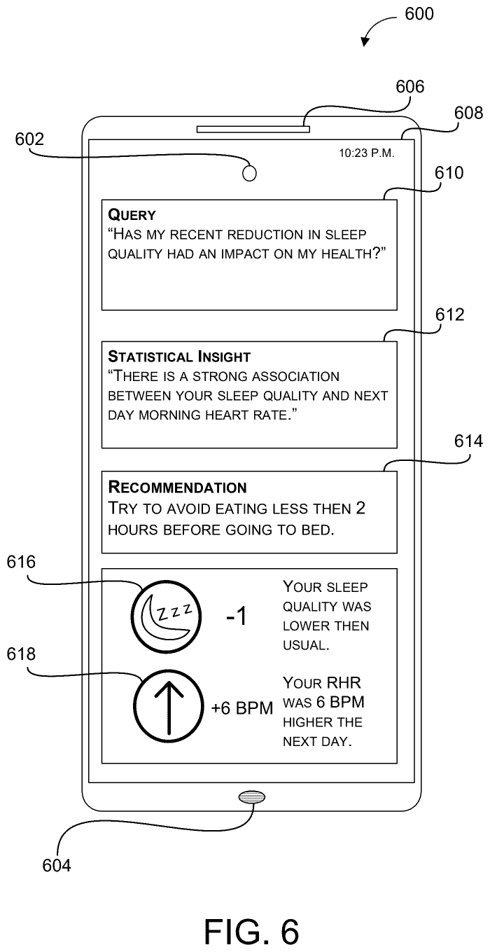

FIG. 6 depicts an example of a statistical insight generation system according to example embodiments of the present disclosure. A computing device 600 can include one or more features and/or capabilities of the computing device 102, the server computing system 130, the model trainer 160, the computing device 300, and/or the computing device 400. Furthermore, the computing device 600 can perform one or more actions and/or operations that can be performed by the computing device 102, the server computing system 130, the model trainer 160, and/or the computing device 300.

As shown in FIG. 6, the computing device 600 includes an imaging component 602, an audio input component 604, an audio output component 606, a display component 608, a query 610, a statistical insight 612, a recommendation 614, a visualization 616, and a visualization 618. The computing device 600 can be configured to perform one or more operations comprising generating one or more key indications based on one or more statistical insights (e.g., statistical insights associated with steps taken by a user).

In this example, the computing device 600 has received the query 610, which is displayed on the display component 608. The query 610 indicates “HAS MY RECENT REDUCTION IN SLEEP QUALITY HAD AN IMPACT ON MY HEALTH?” Based on the query 610, the computing device 600 can determine that the query 610 is seeking to determine whether there are trends in health metrics associated with the user's sleep quality. The computing device 600 can input the query 610 into one or more machine-learned models implemented by the computing device 600. The one or more machine-learned models can be configured to parse the query 610 and generate output comprising one or more objectives, the statistical insight 612, the recommendation 614, the visualization 616, and/or the visualization 618. The one or more machine-learned models implemented by the computing device 400 can generate one or more objectives that indicate which of one or more statistical analysis techniques (e.g., correlation techniques) to perform on one or more health metrics selected from the plurality of health metrics.

Further, the computing device 600 can use the one or more objectives to generate one or more statistical insights. The one or more statistical insights can be based on performing the one or more statistical analysis techniques on the one or more health metrics determined from the query 610 and indicated in the one or more objectives. The one or more statistical analysis techniques can be performed on sleep quality health metrics comprising sleep duration, bedtimes, and/or a number of sleep interruptions per sleeping period. Further, the computing device 600 can process the plurality of health metrics to determine which of the other health metrics has a strong relationship (e.g., the other health metric changes significantly when the sleep quality health metrics change) with the sleep quality health metrics. For example, the computing device 600 can determine that there is a relationship between the sleep quality related health metrics and other health metrics comprising mealtimes, morning heart rates, and/or resting heart rates (“RHRs”).

The statistical insight 612 can be displayed on the display component 608 and can indicate the result of the statistical analysis of the query 610. In this example, the statistical insight 612 indicates “THERE IS A STRONG ASSOCIATION BETWEEN YOUR SLEEP QUALITY AND NEXT DAY MORNING HEART RATE.” The statistical insight 612 can be based on the health metric (e.g., morning heart rate) that was determined to have the strongest relationship with the sleep quality health metrics of the user.

The recommendation 614 can be displayed on the display component 608 and can be based on statistical insight 612. In this example, the recommendation 614 indicates “TRY TO AVOID EATING LESS THAN 2 HOURS BEFORE GOING TO BED.” The recommendation 614 can be based on the statistical insight 612, the one or more objectives, and/or the health data.

The computing device 600 can generate the visualization 616 and/or the visualization 618. The visualization 616 and/or the visualization 618 can be displayed on the display component 608 and can comprise one or more key indications based on the statistical insight 612. In this example, the visualization 616 comprises an image that indicates sleep quality (e.g., an image of a moon) and accompanying text that indicates “YOUR SLEEP QUALITY WAS LOWER THAN USUAL.” Further, the visualization 618 can indicate a change (e.g., an increase) in health metric values comprising a sleep quality metric (e.g., “−1”) that indicates a lower sleep quality and a resting heart rate metric (e.g., beats per minute (BPM) of a resting heart rate) that is shown as a numeric value (e.g., “+6 BPM”). The visualization 618 can include one or more images that indicate the resting heart rate metric (e.g., an image of an arrow corresponding to the resting heart rate metric) that correspond to the numeric value. In this example, the visualization 618 comprises an image of an upward pointing arrow to indicate an increase in the heart rate metric. Further, the visualization 618 comprises text that is associated with the resting heart rate metric and indicates “YOUR RHR WAS 6 BPM HIGHER THE NEXT DAY.”

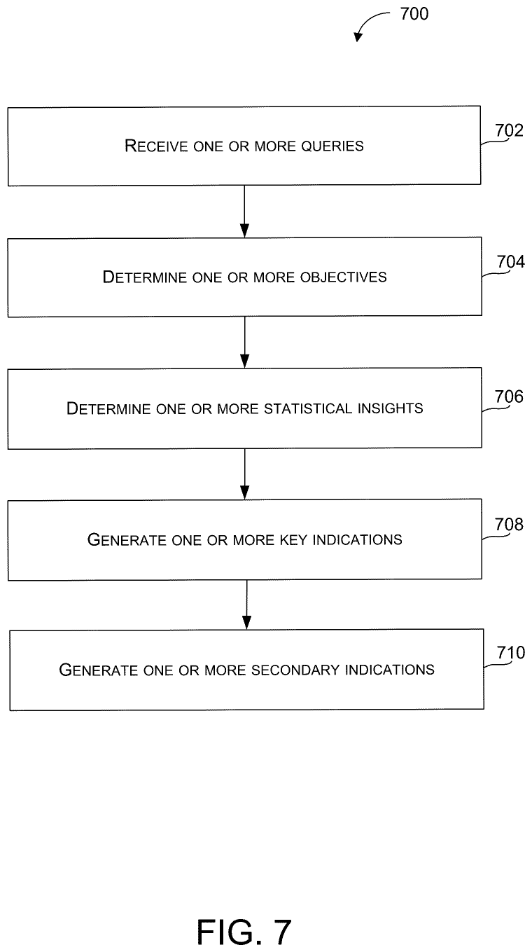

FIG. 7 depicts a flow chart diagram of an example method to perform operations associated with the generation of statistical insights according to example embodiments of the present disclosure. One or more portions of the method 700 can be executed and/or implemented on one or more computing devices or computing systems comprising, for example, the computing device 102, the server computing system 130, the training computing system 150, and/or the computing device 300. Further, one or more portions of the method 700 can be executed or implemented as an algorithm on the hardware devices or systems disclosed herein. FIG. 7 depicts steps performed in a particular order for purposes of illustration and discussion. Those of ordinary skill in the art, using the disclosures provided herein, will understand that various steps of any of the methods disclosed herein can be adapted, modified, rearranged, omitted, and/or expanded without deviating from the scope of the present disclosure.

At 702, the method 700 can include receiving one or more queries. The one or more queries can be associated with health data. Further, the health data can comprise a plurality of health metrics (e.g., health metrics of a user of a computing device). For example, the computing device 102 can access query data that is inputted into a health application that is operational on the computing device 102.

At 704, the method 700 can include determining, based on inputting the one or more queries into one or more machine-learned models, one or more objectives associated with the one or more queries and the plurality of health metrics. For example, the computing device 102 can perform one or more operations comprising using the health data as part of an input to one or more machine-learned models that are configured to receive the input, perform one or more operations on the input, and generate an output comprising the one or more objectives.

The one or more machine-learned models can be configured and/or trained to process and/or parse the health data to determine one or more objectives that may be associated with the one or more queries. For example, the one or more machine-learned models can comprise one or more large language models that are configured and/or trained to determine the portions of one or more queries that refer to objectives (e.g., the portion of the plurality of health metrics that are referred to directly or indirectly in the one or more queries).

At 706, the method 700 can include generating one or more statistical insights based on the one or more objectives. For example, the computing device 102 can use one or more statistical analysis techniques to determine the one or more statistical insights. In some embodiments, the one or more objectives and/or health data can be used as part of an input to one or more machine-learned models that are configured and/or trained to receive the input, perform one or more operations on the input, and generate an output comprising the one or more statistical insights.

At 708, the method 700 can include generating one or more key indications based on the one or more statistical insights and/or the one or more objectives. The one or more key indications can comprise one or more visualizations associated with at least one health metric of the plurality of health metrics. For example, the computing device 102 can include a display device that is used to display the one or more key indications which can comprise a text-based description of the statistical insights and/or a chart with a headline that indicates the contents of the chart which shows some portion of the plurality of health metrics. Further, the computing device 102 can include an audio output device that can be used to generate one or more audio indications that indicate the one or more statistical insights.

In some embodiments, the computing device 102 can generate a recommendation based on the one or more statistical insights. The recommendation can be based on inputting the one or more statistical insights and/or one or more objectives into one or more machine-learned models that are configured and/or trained to generate a recommendation. For example, one or more key statistical insights and/or one or more objectives associated with sleep duration, steps taken, and body mass can be inputted into a machine-learned model that can generate a recommendation to continue taking more than five thousand steps a day and sleeping at least eight hours a night to continue losing body mass.

At 710, the method 700 can include generating one or more secondary indications that can be based on the one or more key indications, can have a higher level of granularity than the one or more key indications, and/or can have a greater depth of information than the one or more key indications. For example, if the one or more key indications comprise weekly health metrics (e.g., steps and heart rate) over a three-month period, the one or more secondary indications can comprise the same key indications on a more granular daily basis and/or over a longer six-month period.

The one or more secondary indications can be generated in response to receiving a request for additional information. For example, a user can interact with (e.g., touch) an interface element of a user interface generated on a computing device (e.g., the interface generated on the computing device 400 that is described with respect to FIG. 4) in order to cause the generation of the one or more secondary indications.

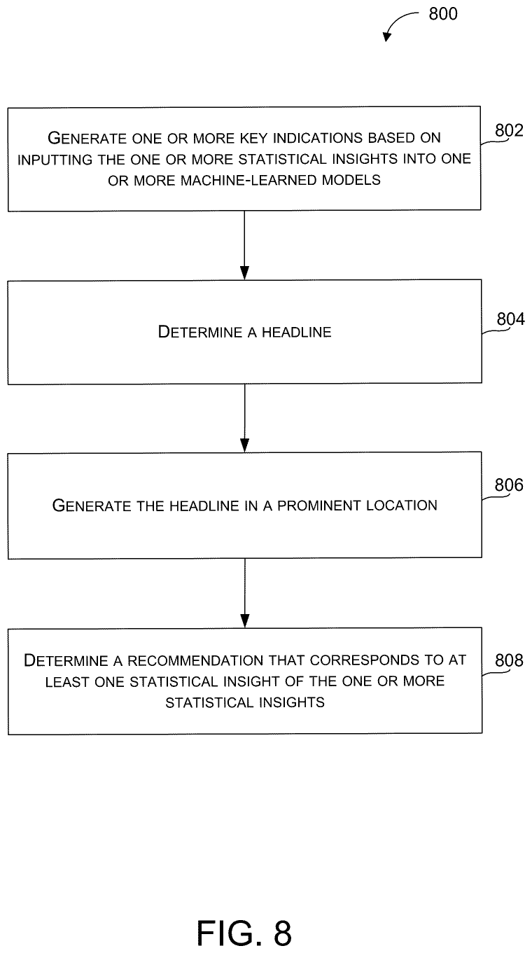

FIG. 8 depicts a flow chart diagram of an example method to perform operations associated with the generation of statistical insights according to example embodiments of the present disclosure. One or more portions of the method 800 can be executed and/or implemented on one or more computing devices or computing systems comprising, for example, the computing device 102, the server computing system 130, the training computing system 150, and/or the computing device 300. Further, one or more portions of the method 800 can be executed or implemented as an algorithm on the hardware devices or systems disclosed herein. In some embodiments, one or more portions of the method 800 can be performed as part of the method 700 that is described with respect to FIG. 7. FIG. 8 depicts steps performed in a particular order for purposes of illustration and discussion. Those of ordinary skill in the art, using the disclosures provided herein, will understand that various steps of any of the methods disclosed herein can be adapted, modified, rearranged, omitted, and/or expanded without deviating from the scope of the present disclosure.

At 702, the method 700 can include generating, based on inputting the one or more statistical insights into the one or more machine-learned models, the one or more key indications. For example, the computing device 102 can implement one or more machine-learned models that are configured to generate the one or more key indications based on input comprising the one or more statistical insights.

At 704, the method 700 can include generating and/or determining a headline that summarizes the one or more statistical insights. For example, the computing device 102 can implement one or more machine-learned models that are configured to generate the headline based on input comprising the one or more statistical insights.

At 706, the method 700 can include generating the headline in a prominent location relative to the one or more visualizations. For example, the computing device 102 can generate the headline immediately above the one or more visualizations or immediately below the one or more visualizations.

At 708, the method 700 can include determining a recommendation that corresponds to at least one statistical insight of the one or more statistical insights. For example, the computing device 102 can implement one or more machine-learned models that are configured to generate the recommendation based on input comprising the one or more statistical insights.

Further to the descriptions above, a user may be provided with controls allowing the user to make an clection as to both if and/or when systems, programs, or features described herein may enable collection of user information (e.g., fitness information, physical exercise activities, and/or a user's preferences), and if the user is sent content or communications from a server. In addition, certain data may be treated in one or more ways before it is stored or used, so that certain information of a user may be removed. For example, a user's identity may be treated so that certain other information associated with the user's identity may not be determined for the user, or a user's geographic location may be generalized where location information is obtained (such as to a city, ZIP code, or state level), so that a particular location of a user cannot be determined. Thus, the user may have control over what information is collected about the user, how that information is used, and what information is provided to the user.

The technology discussed herein makes reference to servers, databases, software applications, and other computer-based systems, as well as actions taken and information sent to and from such systems. The inherent flexibility of computer-based systems allows for a great variety of possible configurations, combinations, and divisions of tasks and functionality between and among components. For instance, processes discussed herein can be implemented using a single device or component or multiple devices or components working in combination. Databases and applications can be implemented on a single system or distributed across multiple systems. Distributed components can operate sequentially or in parallel.

While the present subject matter has been described in detail with respect to various specific example embodiments thereof, each example is provided by way of explanation, not limitation of the disclosure. Those skilled in the art, upon attaining an understanding of the foregoing, can readily produce alterations to, variations of, and equivalents to such embodiments. Accordingly, the subject disclosure does not preclude inclusion of such modifications, variations and/or additions to the present subject matter as would be readily apparent to one of ordinary skill in the art. For instance, features illustrated or described as part of one embodiment can be used with another embodiment to yield a still further embodiment. Thus, it is intended that the present disclosure cover such alterations, variations, and equivalents.

What is claimed is:

1. A computer-implemented method of processing health data, the computer-implemented method comprising:
receiving, by a computing system comprising one or more processors, one or more queries associated with health data comprising a plurality of health metrics;
determining, by the computing system, based on inputting the one or more queries into one or more machine-learned models, one or more objectives associated with the one or more queries;
determining, by the computing system, one or more statistical insights based on the one or more objectives and the health data; and
generating, by the computing system, one or more key indications based on the one or more statistical insights and the health data, wherein the one or more key indications comprise one or more visualizations associated with at least one health metric of the plurality of health metrics.

2. The computer-implemented method of claim 1, wherein the one or more machine-learned models comprise one or more large language models (LLMs) that are configured to determine the one or more objectives based on identifying health-related information in the one or more queries.

3. The computer-implemented method of claim 1, wherein the one or more objectives comprise one or more statistical analysis techniques to perform on one or more health metrics selected from the plurality of health metrics.

4. The computer-implemented method of claim 1, wherein the one or more statistical insights comprise one or more relationships between at least two health metrics of the plurality of health metrics.

5. The computer-implemented method of claim 4, wherein the one or more key indications comprise a description of the one or more relationships between at least two health metrics of the plurality of health metrics.

6. The computer-implemented method of claim 1, wherein the one or more key indications comprise a scatter plot that indicates one or more relationships between at least two health metrics of the plurality of health metrics.

7. The computer-implemented method of claim 1, wherein the one or more statistical insights comprise a trend associated with the at least one health metric of the plurality of health metrics, and wherein the one or more key indications comprise one or more audio indications based on a type of the trend.

8. The computer-implemented method of claim 7, wherein the one or more audio indications comprise a first audio indication based on the type of the trend being an upward trend, a second audio indication based on the type of the trend being a horizontal trend, or a third audio indication based on the type of the trend being a downward trend.

9. The computer-implemented method of claim 1, wherein the generating, by the computing system, one or more key indications based on the one or more statistical insights and the health data comprises:
generating, by the computing system, based on inputting the one or more statistical insights into the one or more machine-learned models, the one or more key indications.

10. The computer-implemented method of claim 1, wherein the generating, by the computing system, one or more key indications based on the one or more statistical insights and the health data comprises:
determining, by the computing system, a headline that summarizes the one or more statistical insights; and
generating, by the computing system, the headline in a prominent location relative to the one or more visualizations.

11. The computer-implemented method of claim 1, wherein the generating, by the computing system, one or more key indications based on the one or more statistical insights and the health data comprises:
determining, by the computing system, a recommendation that corresponds to at least one statistical insight of the one or more statistical insights, wherein the one or more key indications comprise the recommendation.

12. The computer-implemented method of claim 1, further comprising:
in response to receiving a request for additional information, generating, by the computing system, one or more secondary indications that are based on the one or more key indications and have a higher level of granularity than the one or more key indications.

13. The computer-implemented method of claim 1, wherein the plurality of health metrics comprise a plurality of heart rates at a plurality of time intervals, a plurality of body mass values at a plurality of time intervals, a plurality of sleeping hours associated with a plurality of time intervals, or a number of steps associated with a plurality of time intervals.

14. The computer-implemented method of claim 1, wherein the one or more key indications comprise a text-based description of the one or more statistical insights or an audio-based description of the one or more statistical insights.

15. One or more tangible non-transitory computer-readable media storing computer-readable instructions that when executed by one or more processors cause the one or more processors to perform operations, the operations comprising:
receiving one or more queries associated with health data comprising a plurality of health metrics;
determining, based on inputting the one or more queries into one or more machine-learned models, one or more objectives associated with the one or more queries;
determining one or more statistical insights based on the one or more objectives and the health data; and
generating one or more key indications based on the one or more statistical insights and the health data, wherein the one or more key indications comprise one or more visualizations associated with at least one health metric of the plurality of health metrics.

16. The one or more tangible non-transitory computer-readable media of claim 15, wherein the one or more machine-learned models comprise one or more large language models (LLMs) that are configured to determine the one or more objectives based on identifying health-related information in the one or more queries.

17. The one or more tangible non-transitory computer-readable media of claim 15, wherein the one or more objectives comprise one or more statistical analysis techniques to perform on one or more health metrics selected from the plurality of health metrics.

18. A computing system comprising:
one or more processors;
one or more non-transitory computer-readable media storing instructions that when executed by the one or more processors cause the one or more processors to perform operations comprising:
receiving one or more queries associated with health data comprising a plurality of health metrics;
determining, based on inputting the one or more queries into one or more machine-learned models, one or more objectives associated with the one or more queries;
determining one or more statistical insights based on the one or more objectives and the health data; and
generating one or more key indications based on the one or more statistical insights and the health data, wherein the one or more key indications comprise one or more visualizations associated with at least one health metric of the plurality of health metrics.

19. The computing system of claim 18, wherein the one or more machine-learned models comprise one or more large language models (LLMs) that are configured to determine the one or more objectives based on identifying health-related information in the one or more queries.

20. The computing system of claim 18, wherein the one or more objectives comprise one or more statistical analysis techniques to perform on one or more health metrics selected from the plurality of health metrics.
